(19) United States
(12) Patent Application Publication
(10) Pub. No.: US 2012/0010625 A1
(43) Pub. Date: Jan. 12, 2012

(54) REMOTE-CONTROLLED INJECTION APPARATUS FOR INJECTING BONE MATERIAL INTO VERTEBRAL BODY

(76) Inventor: Weixing Shao, Shandong (CN)

(21) Appl. No.: 13/257,601

(22) PCT Filed: Mar. 25, 2010
(86) PCT No.: PCT/CN2010/000371

§ 371 Date: Sep. 20, 2011

(30) Foreign Application Priority Data
Sep. 9, 2009 (CN) 200910018653.1

(51) Int. Cl. A61B 17/58

(52) U.S. Cl. 606/94

(57) ABSTRACT

The present invention relates to two technical programs of a remote-controlled injection device for injecting born materials into centrum, which include a injector with a piston and a precession handspike or slip handspike, a rotational handle or driving handle is fixed at one end of the precession handspike or slip handspike, there is a base in the two programs, and a injector holder is fixed at one end of the base, the said injector is fastened in the injector holder with injector fixed knob by bolt, the said rotational handle or driving handle connects to the handle joint, and there is a fast disassembled connection between handle joint and dynamic joint, the dynamic joint connects with the stepper motor or linear stepper motor, and the projecting shaft of stepper motor or linear stepper motor is coaxial with precession handspike or slip handspike.

### BACKGROUND OF THE INVENTION

The present invention relates to a kind of medical device, especially a remote-controlled injection device for injecting bone materials into the centrum.

Currently, vertebroplasty and replacement need to be operated for the patients who had a Vertebral compression fractures, and in the vertebroplasty surgery, some bone materials such as bone cement need to be injected into the centrum at the fracture location. In the existing technologies, the born materials are injected directly into centrum at the born lesions by injector under the monitoring of radioexamination, for the fluid injection of born materials, the operators should be extremely prudential and careful during injecting operation, once the born materials flowed to the unwanted location by improper operation, it would cause an irreparable medical accident, so a radial monitoring should be needed for reducing accidents during the entire process of born materials injection, therefore, it is necessary for the specific operators in the vertebroplasty surgery to be very close to the radioexamination equipment, which would cause the injury for them, and it is more serious especially for the doctors frequently engaged in such operations. That is the deficiency of the existing technologies.

### BRIEF SUMMARY OF THE INVENTION

The purpose of the present invention is to offer two technical programs of a remote-controlled injection device for injecting born materials into centrum aiming at the shortcomings of existing technologies. In the programs, the specific injection operation will be completed by controlled machine, so operators can be far away from the radioexamination equipment for not only monitoring the injection situation, but also remotely controlling the injection process of injection device, then it will not appear misoperation and can ensure the operators to be avoided damaging by radiation.

The program can be achieved through the following technical measures:

The first technical program:

A remote-controlled injection device for injecting born materials into centrum includes a injector with a piston and a precession handspike, a rotational handle is fixed at one end of the precession handspike, the feature of the program lies in that it includes a base, a injector holder is fixed at one end of the base, and the said injector is fixed in the injector holder, the said rotational handle connects with the handle joint which connects with dynamical joint, and the dynamical joint connects with the projecting shaft of stepper motor which is coaxial with the precession handspike. The specific feature of the program also includes that the said stepper motor is fixed on the motor mounting plate which is installed on the base, and can translate along the base or be locked. The said connection of rotational handle with handle joint is plugging the rotational handle into the handle cavity of handle joint. The connection of handle joint with dynamical joint can be fast disassembly. The said injector holder has a injector fixed knob, which can be used to fasten the injector tightly within the injector holder with bolt.

The second technical program:

A remote-controlled injection device for injecting born materials into centrum includes a injector with a piston and a slip handspike, a driving handle is fixed at one end of the slip handspike, the feature of the program lies in that it includes a base, a injector holder is fixed at one end of the base, and the said injector is fixed in the injector holder, the said driving handle connects with the handle joint which connects with dynamical joint, and the dynamical joint connects with the projecting shaft of linear stepper motor with a thread which is coaxial with the slip handspike, the linear stepper motor is fastened on the base. The specific feature of the program also includes that the said connection of driving handle with handle joint is plugging the driving handle into the handle cavity of handle joint. The connection of handle joint with dynamical joint can be fast disassembly. The said injector holder has a injector fixed knob which can be used to fasten the injector tightly within the injector holder with bolt.

The beneficial effects of the programs can be recognized by the above narration, the precession handspike or slip handspike is coaxial connection with stepper motor or linear stepper motor by handle joint and dynamical joint because the injector is fastened in the injector holder in the two programs. Based on this structure, the injecting action of injector can be controlled by stepper motor or linear stepper motor, and it is easy to achieve the remote-controlled of stepper motor or linear stepper motor for person, so the operators can be far away from the injection site to implement the injection in front of the monitoring display of radioexamination equipment, that ensures the accuracy of the operation and avoids the misoperation, and therefore greatly enhances the quality of surgery. The control for stepper motor or linear stepper motor can also achieve some operations such as fast moving forward, fast moving back, injection, stop and clearing, and the dose can be displayed in real time, and it can be set in advance. Thus, the present invention has substantive features and progress compared with existing technologies, it is also obvious for its beneficial effects of implementation.

### BRIEF DESCRIPTION OF THE DRAWINGS

FIG. 1 is the partly sectional structure view of the first embodiment of the invention.

FIG. 2 is the top structure view of the first embodiment of the invention.

FIG. 3 is the partly sectional structure view of the second embodiment of the invention.

Where, 1 is base, 2 is injector, 3 is piston, 4 is precession handspike, 5 is injector holder, 6 is injector fixed knob, 7 is hand cavity, 8 is rotational handle, 9 is handle joint, 10 is dynamical joint, 11 is stepper motor, 12 is motor mounting plate, 13 is slip handspike, 14 is driving handle, 15 is linear stepper motor.

### DETAILED DESCRIPTION OF THE INVENTION

In order to clearly explain the technical features of the programs, the following will describe them with two embodiments and their drawings.

The first technical program:

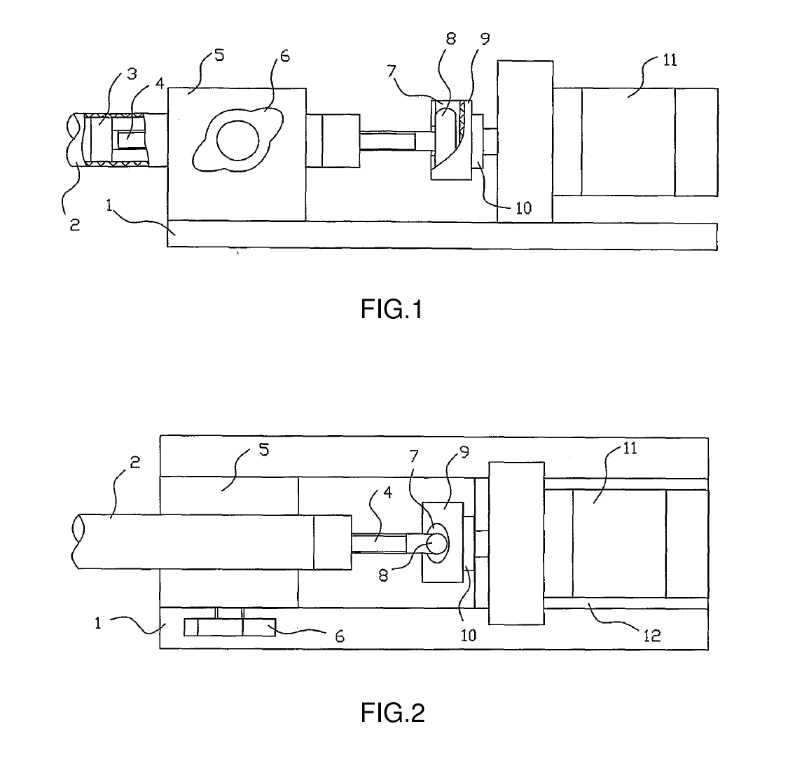

It can be seen from the FIG. 1 and FIG. 2, the remote-controlled injection device for injecting born materials into centrum of the invention includes a base 1, and a injector holder 5 fastened at one end of the base 1, the injector 2 with piston 3 and precession handspike 4 is tightly fastened in the injector holder 5 with injector fixed knob 6 by bolt. The rotational handle 8 at one end of precession handspike 4 connects with handle joint 9 through rotational handle 8 plugging into the handle cavity 7 of handle joint 9. There is a fast disassembled connection between handle joint 9 and dynamic joint 10, the structure of fast disassembly is various in the existing technologies, any one can be used. Dynamic joint 10 connects with the projecting shaft of the stepper motor 11, and the projecting shaft of stepper motor 11 is coaxial with the precession handspike 4. the said stepper motor 11 is fastened on the motor mounting plate 12 which is fixed on the base 1 and can translate along the base 1 or be locked.

The second technical program:

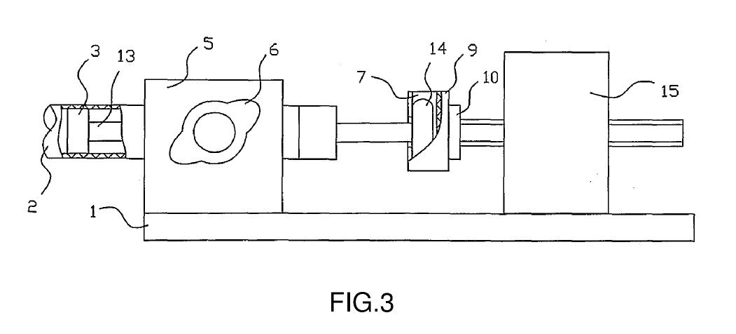

It can be seen from the FIG. 3, the remote-controlled injection equipment for injecting born materials into centrum of the invention includes a base 1, and a injector holder 5 fastened at one end of the base 1, the injector 2 with piston 3 and precession handspike 4 is tightly fastened in the injector holder 5 with injector fixed knob 6 by bolt. The driving handle 14 at one end of slip handspike 13 connects with handle joint 9 through driving handle 14 plugging into the handle cavity 7 of handle joint 9. There is a fast disassembled connection between handle joint 9 and dynamic joint 10, the structure of fast disassembly is various in the existing technologies, any one can be used. Dynamic joint 10 connects to the projecting shaft with thread of the linear stepper motor 15, and the projecting shaft with thread of linear stepper motor 15 is coaxial with that of the slip handspike 13, and the linear stepper motor 15 is fastened on the base 1.

What is claimed is:

1. A remote-controlled injection device for injecting born materials into centrum includes a injector with a piston and a slip handspike, a rotational handle is fixed at one end of the precession handspike, wherein there is a base, and a injector holder fastened at one end of the base, the said injector is fastened in the injector holder, the said rotational hand connects with the handle joint which connects with the dynamical joint, and the dynamical joint connects with the projecting shaft of stepper motor which is coaxial with the precession handspike.

2. The injection device of claim 1, wherein the said stepper motor is fixed on the motor mounting plate which is fixed on the base and can translate along the base 1 or be locked.

3. The injection device of claim 1, wherein the connection of rotational handle with handle joint is accompleted by plugging rotational handle into handle cavity of handle joint.

4. A remote-controlled injection device for injecting born materials into centrum includes a injector with a piston and a slip handspike, a driving handle is fixed at one end of the slip handspike, wherein there is a base, and a injector holder fastened at one end of the base, the said injector is fastened in the injector holder, the said driving handle connects with the handle joint which connects with the dynamical joint, and the dynamical joint connects to the projecting shaft with thread of linear stepper motor which is coaxial with the slip handspike, the linear stepper motor is fixed on the base.

5. The injection device of claim 4, wherein the connection of driving handle with handle joint is accompleted by plugging driving handle into handle cavity of handle joint.

6. The injection device of claim 1 or 4, wherein the connection of the said handle joint with dynamical joint can be disassembled quickly.

7. The injection device of claim 1 or 4, wherein the injector holder has a injector fixed knob which can be used to tightly fasten the injector within the injector holder with bolt.
